(19) United States
(12) Patent Application Publication
(10) Pub. No.: US 2006/0110371 A1
(43) Pub. Date: May 25, 2006

(54) Control of indoleamine 2,3 deoxygenase expression and activity

(75) Inventors: Matthew Albert, Paris (FR); Deborah Braun, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: INSTITUT PASTEUR, Paris (FR)

(21) Appl. No.: 11/251,877

(22) Filed: Oct. 18, 2005

Related U.S. Application Data
(60) Provisional application No. 60/629,896, filed on Nov. 23, 2004.

(51) Int. Cl. A61K 35/14; C12N 5/08

(52) U.S. Cl. 424/93.7; 435/372

(57) ABSTRACT

The present invention relates to controlling and/or manipulating of dendritic cells by controlling the expression and activity of indoleamine 2,3 deoxygenase expression and activity.

### CROSS-REFERENCE TO RELATED APPLICATIONS

The present application claims priority to U.S. provisional application U.S. 60/629,896, filed on Nov. 23, 2004, the entirety of which is incorporated herein by reference.

### BACKGROUND OF THE INVENTION

1. Field of the Invention

The present invention relates to controlling and/or manipulating of dendritic cells by controlling the expression and activity of indoleamine 2,3 deoxygenase expression and activity.

2. Description of the Background

Two factors contribute to the difficulty in determining how the immune system can eradicate tumors in humans. First, it has been difficult to identify examples of naturally occurring tumor immunity to study. Second, cytolytic T lymphocytes (CTLs) have not been found to be expanded in patients with active tumors, even when the tumors express tumor-specific antigens such as the melanoma MAGE and MART (Schuler, G. & Steinman, R. M. Dendritic cells as adjuvants for immune-mediated resistance to tumors. J Exp Med 186, 1183-7 (1997)). In fact, the latter issue may naturally follow from the first as scientists have only been looking at patients with cancer. In earlier studies, patients with breast and ovarian cancer that go on to develop paraneoplastic cerebellar degeneration (PCD) were evaluated. These individuals provided important examples of naturally occurring tumor immunity in humans, offering us insight into important scientific and medically relevant issues. PCD is associated with breast and ovarian tumor cell expression of neuron-specific proteins. It has been demonstrated that tumor-specific CTLs are important in mediating this tumor immunity (Albert, M. L. et al. Tumor-specific killer cells in paraneoplastic cerebellar degeneration. Nat Med 4, 1321-4 (1998); Darnell, R. B. Proc Natl Acad Sci U S A 93, 4529-36 (1996))

Cytotoxic T lymphocytes are an important component of the adaptive immune response. They destroy infected cells and are considered critical for the eradication of cells on their way toward malignant transformation (Pamer, E. & Cresswell, P Annu Rev Immunol 16, 323-58 (1998)). To become an effector cell and thus perform these tasks, CTLs must first be activated by an antigen presenting cell (APC) expressing MHC class I/peptide complexes on its' cell surface. Dendritic cells (DCs) are considered to be the only APC capable of priming naive T cells, and are also potent stimulators of recall responses (Banchereau, J. & Steinman, R. M. Dendritic cells and the control of immunity. Nature 392, 245-52 (1998)). Briefly, DCs exist in the periphery as immature cells where they serve as ‘sentinels,’ responsible for capturing antigen. Upon maturation/activation, DCs migrate to the draining lymph organs, where they may initiate immune responses. This ability to traffic out of peripheral tissue with captured antigen, and enter the afferent lymph is unique to the DCs, making them the appropriate carrier of tissue-restricted antigen to lymph organs for the initiation of tumor-immunity.

To understand naturally occurring tumor immunity, it has been found that the peripheral tissue DC, exemplified by the immature DC, can phagocytosing apoptotic tumor cells (Albert, M. L. et al J Exp Med 188, 1359-68 (1998); Albert, M. L., Sauter, B. & Bhardwaj, N Nature 392, 86-9 (1998)). Following antigen acquisition, we envision that DCs migrate to the draining lymph nodes where T cells are engaged, resulting in the cross-priming of tumor-reactive CTL. Such a pathway has now been demonstrated both in vitro using human primary DCs and T cells (Albert, M. L. et al. Tumor-specific killer cells in paraneoplastic cerebellar degeneration. Nat Med 4, 1321-4 (1998); Albert, M. L., Jegathesan, M. & Darnell, R. B. Nat Immunol 2, 1010-7. (2001).) and in vivo using mouse models (Heath, W. R. & Carbone, F. R. Nat Rev Immunol 1, 126-34 (2001); Huang, A. Y. et al. Science 264, 961-5 (1994)), and it has served as a basis for DC-based immunotherapy trials in the area of breast cancer, prostate cancer and melanoma (Neidhardt-Berard, E. M., Berard, F., Banchereau, J. & Palucka, A. K. Breast Cancer Res 6, R322-8 (2004); Orange, D. E. et al. Prostate Cancer Prostatic Dis 7, 63-72 (2004)). Specifically, ours, and the proposed immunotherapy protocols of others have paralleled the defined physiologic event: immature DCs are prepared from patients and cultured with antigen in order to charge them with immunologic epitopes (we have employed apoptotic cells, but the insights gleaned from the proposed work may also impact work with peptide, viral vaccines and exosomes); and upon ex vivo maturation, the DCs are re-infused in hopes of activating tumor-specific CTLs (Nestle, F. O., Banchereau, J. & Hart, D. Nat Med 7, 761-5 (2001)). While we are carrying out one such study using autologous DCs cross-presenting apoptotic prostate tumor cells for the stimulation of tumor-reactive T cells in prostate cancer patients (collaboration with R. Darnell at The Rockefeller University Hospital), it remains critical that we also work to better define mechanisms that will improve upon this therapeutic modality.

While much of our work has focused on the activation of CTLs, it is now known that DCs may also present exogenous tumor antigen in a manner that results in the tolerization of T cells. This important phenomenon was first demonstrated in a model for peripheral tolerance using a neo-self antigen, chicken ovalbumin protein (OVA), expressed specifically in the b cells of the pancreas (Kurts, C. et al. J Exp Med 184, 923-30 (1996)). When OVA-specific, MHC I-restricted TCR transgenic CD8+ T cells were adoptively transferred into these mice, the T cells accumulated and expanded in the draining lymph node. These T cells were responding to antigen presented by bone marrow derived cells and not to the islet cells themselves. Following the observed proliferation, T cells died via apoptosis, suggestive of peripheral tolerance or deletion of self-reactive CTLs (Kurts, C. et al. J Exp Med 184, 923-30 (1996); Heath, W.R., J Exp Med 187, 1549-53 (1998); Kurts, C., J Exp Med 188, 415-20 (1998); Kurts, C. et al. Nature 398, 341-4 (1999)). In contrast, when OVA-specific MHC I- and MHC II-restricted TCR transgenic T cells were both transferred into the same mice, now the CD8+ T cells became effector cells and lysed the OVA-expressing islet cells resulting in diabetes (Kurtis et al J Exp Med 186, 2057-62 (1997); Bennet et al J Exp Med 186, 65-70 (1997)). It was therefore proposed that a bone marrow derived cell captures antigens for both MHC class I and class II presentation, migrates to the lymph node and cross-presents the antigen to CD4+ and CD8+ T cells.

We have developed an in vitro system to study the immunologic switch between T cell activation vs. tolerance. As has been observed in vivo, we defined a requirement for CD4+ T helper cells for the activation of CTLs via the cross-presentation pathway. We have also shown that the absence of CD4 helper cells triggers a tolerance pathway-antigen-specific CD8+ T cells undergo 4-6 rounds of cell division and die an apoptotic cell death (Albert, M. L., Jegathesan, M. & Darnell, R. B. Nat Immunol 2, 1010-7. (2001)). This model system has allowed us to dissect the cellular and molecular events regulating cross-presentation. As discussed below, these findings will be applied toward the study of tumor immunity and understanding tumor-mediated immunosuppression.

Regulation of IDO expression and activity is poorly understood. Indoleamine 2,3-dioxygenase (IDO) is an enzyme involved in tryptophan catabolism. Initially, it was characterized for its role in antimicrobial resistance: by actively depleting tryptophan, essential for micro-organisms growth, both within the infected cell and in the surrounding milieu, IDO serves to suppress growth of invasive bacterial. More recently, studies by David Munn and Andrew Mellor have established a role for IDO in maternal tolerance and possibly more general aspects of T cell tolerance (Mellor et al Adv Exp Med Biol 527, 27-35 (2003); Munn et al Science 281, 1191-3 (1998)). IDO expression has been reported in placental trophoblasts and IFN-g activated antigen presenting cells (including macrophages and DCs), reflecting its counter-inflammatory role. The precise mechanism of action remains unknown, but may include the depletion of tryptophan or the production of cytolytic catabolites such as kynurenine, which has been shown to induce T cell apoptosis.

An exciting advance for this field has been the discovery that IDO is initially expressed as a pro-enzyme. While the biochemical signal responsible for activation are not known, Grohmann and colleagues have shown that reverse signaling via B7 (CD80/CD86) is responsible for IDO activation (Grohmann et al, Nat Immunol 3, 1097-101 (2002); Fallarino et al, Nat Immunol, 4, 1206-12 (2003)). Furthermore, CD40 engagement seems to shut off IDO enzymatic activity. Together, this suggests that T cell/DCs interactions may regulate IDO activity. We have recently demonstrated that IDO is one of the most highly upregulated genes during DC and describe herein the implications of this finding for DC immunotherapy.

In addition to DCs acting on T cells in a manner that results in tolerance, the putative target cell (in this case the breast tumor cell) may actively inhibit the adaptive immune response. Several such mechanisms have been reported, including expression of FasL and Spi-6 by tumor cells, which act to kill tumor reactive T cells or inhibit their ability to kill via Granzyme B, respectively (Medema et al, Proc Natl Acad Sci USA 98 111515-20 (2001); Botti et al, Clin Cancer Res 10, 1360-5 (2004). Recently, IDO expression by tumor cells was added to the list of mechanisms by which malignancies may evade immunity. It was demonstrated that 10/10 cervical carcinomas, 8/10 ovarian carcinomas and 3/10 breast carcinomas were found to express IDO30. Moreover, in mouse studies where tumors were transfected with DNA constructs expressing IDO, the tumors were less susceptible to CTL-mediated tumor immunity; and administration of 1-methyl tryptophan (1-MT) recovered the ability to establish protective immunity. With respect to breast carcinoma, another group demonstrated that the cell line MDA-MB-231 but not MCF-7 expressed IDO, leading them to suggest that estrogen receptor (ER) negativity may correlate with IDO activity and immune evasion (Travers et al Biochim Biophys Acta 1661, 106-12 (2004)). Given the possibility that IDO expression in ER- tumors may contribute to the poor prognosis of patients with such malignancies, it is imperative that this correlation be rigorously tested using primary samples. As discussed below, we will also analyze the data, evaluating IDO expression as an independent prognostic indicator for recurrence or survival.

While immunotherapy strategies hold much promise, a serious limitation in the development of such therapeutic modalities has been the tumors' ability to evade the immune system. Indeed, these strategies may be inherent to the pathogenesis of disease. Additionally, as increasing numbers of therapeutic approaches are being considered (e.g. angiogenesis inhibitors, novel chemotherapeutics), it is important to be able to pre-select patients for whom such a treatment would be successful.

Typically, dendritic cells used in immuno-therapeutic trials and/or cancer immuno-therapeutic trials are matured with a cocktail of cytokines including prostaglandin E2 (PGE2). In addition, as described above, IDO is another enzyme which has been shown to be involved in some general aspects of T cell tolerance.

### SUMMARY OF THE INVENTION

The inventors have discovered a two step mechanism for activating the IDO enzyme. While PGE2 by itself induces expression of IDO, a second signal through TNFR and/or TLRs facilitates IDO enzymatic activation.

One embodiment of the present invention is a method to modulate the maturation of a dendritic cell. By modulation of the maturation of a dendritic cell, we mean the stabilization of a cell type capable of inducing tolerance. In other terms used in the field, this may embody the stabilization of a so-called ‘semi-mature’ dendritic cell; or the ‘skewing’ of the dendritic cell towards an alternate maturation program that facilitates release of regulator or tolerogenic cytokines (and/or facilitates induction of regulatory or tolerogenic cell types)

Another embodiment of the present invention is to a method of providing a dendritic cell composition to an individual, comprising contacting the dendritic cell composition with at least one inhibitor in an amount sufficient such that IDO modulates the maturation process of the dendritic cell to stabilize a phenotype that drives CD+8 T cell tolerance, wherein the at least one inhibitor is selected from the group consisting of an IDO inhibitor, a PEG2 inhibitor, a TNFR inhibitor, a TLR inhibitor and mixtures thereof; and thereafter providing the dendritic cell composition to the individual.

One embodiment of the present invention is a mature dendritic cell comprising a reduced level of IDO activity relative to a normal dendritic cell and which can abolish T cell tolerance in an individual and which drives CD8+ T cell activation.

Another embodiment of the present invention is a method of obtaining a mature dendritic cell, comprising contacting the dendritic cell with at least one inhibitor in an amount sufficient to modulate the maturation of the dendritic cell, wherein the at least one inhibitor is selected from the group consisting of an IDO inhibitor, a PEG2 inhibitor, a TNFR inhibitor, a TLR inhibitor and mixtures thereof.

Another embodiment of the present invention is a method for identifying an IDO inhibitor, comprising contacting a cell with a substance, measuring the level of at least one of IDO expression and IDO activity; and comparing the level of at least on IDO expression and IDO activity in the cell contacted with the substance to a cell not contacted with the substance; wherein a decrease in at least one of IDO expression and IDO activity in the cell contacted with the substance relative to the cell not contacted with the substance indicates that the substance is an IDO inhibitor.

Another embodiment of the present invention is a method of predicting a tumors ability to evade an individual's immune system, comprising measuring the level of IDO activity in a cell isolated from the tumor, wherein a decrease in IDO activity relative to a control tumor cell which cannot evade an individuals immune system indicates that the tumor can evade the individual's immune system.

Another embodiment of the present invention is a method of treating an immunological disorder in an individual, comprising administering at least one IDO inhibitor to an individual in need thereof, in an amount sufficient to block the negative effects of PGE2 stimulation of IDO, stimulate dendritic cell migration, and treat the immunological disorder.

Another embodiment of the present invention is a method of treating an immunological disorder in an individual, comprising administering the mature dendritic cell according to the invention to an individual in need thereof, in an amount sufficient to block the negative effects of PGE2 stimulation of IDO, stimulate dendritic cell migration, and treat the immunological disorder.

Another embodiment of the present invention is a method of treating a tumor in an individual, comprising administering at least one IDO inhibitor to an individual in need thereof, in an amount sufficient to block the negative effects of PGE2 stimulation of IDO, stimulate dendritic cell migration, and treat the tumor in the individual.

Another embodiment of the present invention is a method of treating a tumor in an individual, comprising administering the dendritic cell according to the invention, in an amount sufficient to block the negative effects of PGE2 stimulation of IDO, stimulate dendritic cell migration, and treat the tumor in the individual.

Another embodiment of the present invention is a method of treating an inflammatory skin condition in an individual, comprising topically applying a composition on at least one skin area of the individual, wherein the composition comprises at least one of PGE2, PGE-2 agonist, and EP2, in an amount sufficient to upregulate IDO and treat the inflammatory skin condition.

Another embodiment of the present invention is a method of treating an inflammatory skin condition in an individual, comprising administering the mature dendritic cell according to the invention, in an amount sufficient to treat the inflammatory skin condition.

Another embodiment of the present invention is a method of treating an inflammatory lung condition in an individual, comprising administering an aerosol composition into a lung area of the individual, wherein the composition comprises at least one of PGE2, PGE-2 agonist, and EP2, in an amount sufficient to upregulate IDO and treat the inflammatory lung condition.

Another embodiment of the present invention is a method of treating an inflammatory lung condition in an individual, comprising administering the mature dendritic cell according to the invention into a lung area of the individual, in an amount sufficient to treat the inflammatory lung condition.

Another embodiment of the present invention is a method of treating a tumor in an individual, comprising administering at least one IDO inhibitor to the individual in an amount sufficient to treat the tumor in said individual, wherein the tumor comprises an overexpressed COX-2 gene.

Another embodiment of the present invention is a kit, comprising kynurenine and one or more reagents suitable for detecting a tumor's ability to evade an individual's immune system.

Another embodiment of the present invention is a kit, comprising at least one purified oligonucleotide which hybridizes with a polynucleotide encoding IDO, and at least one reverse transcriptase reagent suitable for detecting the mRNA level of IDO in a sample.

Another embodiment of the present invention is a technical platform, comprising a sample from a patient's fluid or a solid tumor, a device for quantifying IDO activity, and at least one inhibitor for the stabilizing process of mature dendritic cells having a phenotype that drives CD+8 T cell tolerance, wherein the inhibitor is an IDO inhibitor, a PGE2 inhibitor, a TNFR inhibitor, a TLR inhibitor, or a combination thereof.

The above objects highlight certain aspects of the invention. Additional objects, aspects and embodiments of the invention are found in the following detailed description of the invention.

### BRIEF DESCRIPTION OF THE DRAWINGS

A more complete appreciation of the invention and many of the attendant advantages thereof will be readily obtained as the same becomes better understood by reference to the following Figures in conjunction with the detailed description below.

FIG. 1: shows the detection of IDO mRNA by qRT-PCR.

FIG. 2: shows a correlation between HPLC and colorimetric assays for analysis of kynurenine concentrations. (A) shows HPLC profiles of iDC and mDC (TNFα+PGE2) culture supernatants. (B) shows a standard curve obtained for a representative experiment using data obtained from a colorimetric assay using kynurenine concentrations between 0 and 100 mm with a resulting linear extrapolation of R²=0.9995. (C) shows kynurenine levels for 4 (run from a single donor) of 26 samples measured in parallel by HPLC and colorimetric assay with standard deviation obtained from two independent wells for each sample. (D) shows a correlation plot for all 26 samples analyze by HPLC and colorimetric assay with a linear correlation curve having a ρ=0.755 indicating that there is no significant departure from linearity.

FIG. 3: shows an overview of defining the distinct outcomes of Antigen Cross-presentation

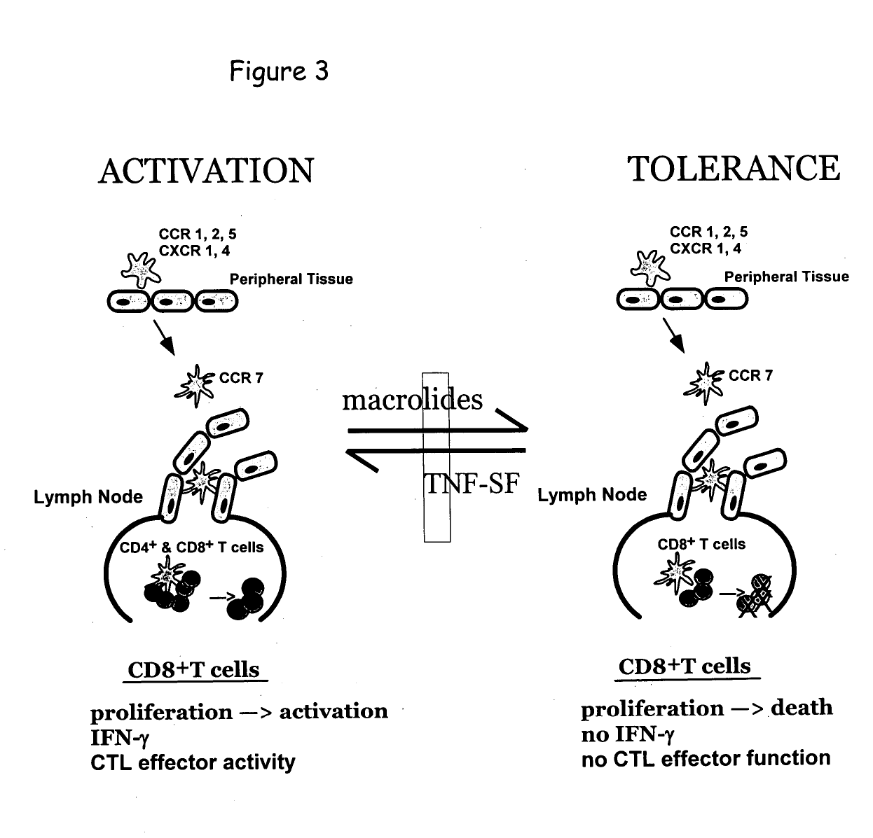

FIG. 4: shows the regulation of indoleamine 2,3 dioxygenase during DC maturation.

FIG. 5: shows the adenylate cyclase activator forskolin mimics PGE2 and induces IDO expression.

FIG. 6: shows that the peak IDO activity is at 48 hr DC maturation.

FIG. 7: shows a kinetic study of IDO activity.

FIG. 8: shows that PGE-2 acts via four receptors—EP 1-4 The Ga-coupled receptors EP2 and EP4 are expressed on myeloid DCs and act via cAMP.

FIG. 9: shows that the adenylate cyclase activator forskolin mimics PGE2 and induces IDO expression.

FIG. 10: shows the adenylate cyclase inhibitor SQ22536 blocks IDO expression/activity

FIG. 11: shows that the PGE2-receptor EP2 is responsible for stimulating IDO expression in DCs.

FIG. 12: shows that 1-methyl tryptophan (1-MT) inhibits IDO activity in mature DCs.

FIG. 13: shows Anti-IDO Western Blot

FIG. 14: shows data extracted from Affymetrix gene array studies (U133A chips) for four experiments As described in the examples, immature dendritic cells (iDC) were differentiated from monocyte precursors using GM-CSF and IL-4. These cells were then exposed to TNFα and PGE2 for 36 hrs in order to generate mature dendritic cells (mDC). The raw signal intensity reflects the relative expression of IDO (lines ranging from 0 for immature DCs to a range of approx. 4000-11000 for mature DCs) and GAPDH (lines remaining relatively stable from immature to mature DCs and falling in the range of approximately 5000-7000). Each line indicates an independent donor.

FIG. 15: shows that the maturation stimulus used influences IDO expression and activity. Human monocyte-derived iDCs were cultured in media alone, or exposed to distinct maturation stimuli as indicated. (A) The relative expression levels of IDO were measured using quantitative RTPCR. IDO expression is represented as a ratio of IDO/GAPDH as compared to iDCs (set to a value of 1.0 in order to normalize the data). Each bar corresponds to the mean of all donors assayed (n=1-7). Error bars indicate standard error of the mean (SEM). Non-normalized data is reported in Table 1. (B) IDO protein was detected by Western Blot in cell extracts using a polyclonal rabbit anti-human IDO Ab. The cell number from which the protein was derived was normalized prior to loading the gel; and Ponceau Red staining of the membrane confirmed that equivalent protein content was being analyzed (data not shown). (C) Following the different culturing conditions, DCs were washed well and incubated for 4 hrs in HBSS containing 100 μM tryptophan. Supernatants were harvested and the concentration of kynurenine was determined. The mean concentration of kynurenine, as measured by HPLC, is represented (n=1-6). Error bars indicate standard error of the mean (SEM). Numeric values and the range observed in different donors are reported in Table 1.

FIG. 16: shows that PGE2 triggers the transcription of IDO mRNA. (A, B) IDO mRNA expression in DCs stimulated with TNFα, PGE2 or a combination of both stimuli was monitored by qRT-PCR. The PCR amplification curves from a representative experiment, performed in duplicate, are displayed (A). The same data is reported as a ratio of IDO/GAPDH, again normalized to expression in iDCs (set to a value of 1.0). The inset in (B) represents the level of IDO mRNA as a function of the dose of PGE2. (C) DCs exposed to the conditions described above were monitored for their IDO activity. Kynurenine production was measured using a colorimetric assay; known concentrations were used for the establishment of a standard curve (see FIG. 2B). Values are the mean of triplicate wells and error bars indicate standard deviation. Data in FIG. 3 is representative of 4 experiments.

FIG. 17: shows that TNF-R and TLR engagement triggers IDO enzymatic activity. DCs were prepared as described in the Examples, with maturation stimuli consisting of TNFα, LPS or SAC, either in the presence or absence of PGE2. (A) IDO protein expression was detected by Western blot and (B) enzymatic activity was quantified by the colorimetric assay for kynurenine, as described in the Methods section.

FIG. 18: shows that PGE2 acts via EP2 to stimulate IDO expression. (A) The present inventors monitored EP2 and EP4 mRNA expression in DCs exposed to TNFα, PGE2 or a combination of both stimuli. TBP was used as a reference for our qPCR studies as its expression levels matched that of the EP-Rs. White bars represent EP2 levels; black bars indicated EP4 expression. Of note, no message could be detected for EP1 and EP3 by qPCR (data not shown). (B) PGE2 was replaced by different EP agonists during DC maturation and IDO activity was assessed after 48 hrs. L-335677, Butaprost, L-826266 and L-161982 were used to stimulate EP1-4, respectively. All were used at a concentration of 50 μM. In similar experiments, sulprostone was used as an EP1>>EP3 agonist and 19R-hydroxy PGE2 as an EP2 agonist, with similar results (data not shown).

FIG. 19: shows that PGE2 signals via cAMP and PKA to trigger IDO. (A) The adenylate cyclase activator forskolin mimics PGE2 activation of EP2/4 and induces IDO activity in the presence of TNFα. MoDCs were matured during 48 hrs in the presence of increasing doses of forskolin +/− TNFα. IDO activity was assessed spectrophotometrically after 4 hrs at the end of the culture. (B) The adenylate cyclase inhibitor SQ22536 and the PKA inhibitor H-89 block IDO expression. Monocyte-derived DCs were cultured in the presence of PGE2 or forskolin, in addition to TNFα for 48 hrs. The effect of inhibiting adenylate cyclase during this culture using SQ22536, as well as the effect of the PKA inhibitor H-89, were assessed on IDO mRNA levels.

### DETAILED DESCRIPTION OF THE INVENTION

Unless specifically defined, all technical and scientific terms used herein have the same meaning as commonly understood by a skilled artisan in enzymology, biochemistry, cellular biology, molecular biology, and the medical sciences.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

The present inventors have discovered the physiologically relevant pathway for inducing the tolerogenic enzyme IDO. This discovery permits overcoming dendritic cell mediated or tumor mediated tolerance in cancer patients. Further, triggering IDO expression and the tolerization of self-reactive, alergan-reactive or graft-reactive T cells in the respective patient population.

The present inventors analyzed the transcription profile of DC matured in the presence of TNFα and PGE2 and have discovered that strong up-regulation of IDO, an enzyme involved in tryptophan catabolism and implicated in both maternal and T cell tolerance. The inventors further discovered that the conditions of DC maturation dictate the activity of IDO. Thus, the inventors describe that PGE2 induces expression of IDO but activation of the enzyme is mediated by a second signal through TNFR or TLRs. Further, the inventors also describe that PGE 2 may act through two known GPCRs, EP2 and EP4 to trigger the activation of adenylate cyclase.

Dendritic cells are known in the art and can comprise cells isolated directly from the hematopoietic system, e.g., peripheral blood, bone marrow, organs or tissues; or obtained from cell cultures of peripheral blood, bone marrow, organs or tissues or isolated CD34+ stem cells collected from peripheral blood or bone marrow which express CD83 constitutively or following culture and maturation (or a level of maturation). Dendritic cells can be cultured in any conventional medium commonly used in the art, and can include cytokines, if desirable, such as GMCSF, IL4, IL3, and IL10.

By mature dendritic cell it must be comprised the population of cells that have been rendered capable of processing antigen (e.g. acidification of lysosomes, see Mellman); upregulating CCR7, which will permit dendritic cell entry into the T cell areas of lymphoid organs; or cell types expressing levels of co-stimulatory molecules that permit maximal activation in an allogeneic mixed lymphocyte reaction. Importantly, the mature dendritic cell has the capacity to induce either CD8 T cell activation (namely, proliferation, IFN-γ production and CTL function) or CD8 T cell tolerance (proliferation leading to death, no IFN-γ production, no CTL function) depending on the environment leading to DC maturation and/or the environment of the lymph node.

Methods to obtain mature dendritic cells are described, for example in U.S. Pat. No. 6,602,709 Albert Matthew et al. and in Romani, et al., in “Generation of Mature Dendritic Cells from Human Blood: an Improved Method with Special Regard to Clinical Applicability,” J. Immunological Methods, vol. 196, pp. 137-151, 1996, the contents of which are incorporated herein by reference.

In one embodiment, the present invention provides tools and methods for controlling the maturation of dendritic cells by controlling the expression and/or activity of indoleamine 2,3 deoxygenase such that maturation process is skewed to stabilize a phenotype that drives CD+8 T cell tolerance. Such modulation of dendritic cell maturation can be useful for providing immunotherapies to individuals in need of such treatment. For example, in one embodiment, the immunotherapy can be applied to the treatment of tumors.

The mature dendritic cells of the present invention are able to abolish T cell tolerance. Further, the activity of IDO in the mature dendritic cells is inhibited relative to a non-modulated dendritic cell. The mature dendritic cells of the present invention also have a phenotype that drives CD8+ T cell tolerance. Thus, one embodiment of the present invention is to a mature dendritic cell with one or more of these functional and structural features.

Modulating dendritic cell maturation can be accomplished, in one embodiment, by inhibiting the expression and/or activity of IDO (Indolemine 2, 3 dioxygenase). In another embodiment, the modulation and/or control of dendritic cell maturation can be accomplished by inhibiting PEG2 (prostaglandin). Inhibitors of TNFR and/or TLR can also be used. In another embodiment, IDO activity can be inhibited by genetic engineering such as modifications in the gene coding, non-coding, and/or control sequences of the IDO gene. Combinations of these approaches are also provided herein.

The IDO, PEG2, TNFR or TLR inhibitors as used in the present invention can be those known in the art. In addition, inhibition of expression and/or activity can be accomplished using small molecules, ribozymes, antisense, antibodies, and small interfering RNA (siRNA) molecules specific for the target. Combinations of these are also provided herein.

An IDO inhibitor can further be chosen from the (D) isomer analogue of tryptophan and derivatives thereof, including, for example, 1-methyl-(D,I)-tryptophan, β-(3-benzofuranyl)-DL-alanine (1-MT), β-[3-benzo(b)thienyl]-(D,L)-alanine, and 6-nitro-(D,L)-tryptophan. Combinations of these inhibitors are also provided herein.

The ability to control and/or modulate the maturation of dendritic cells has several advantages. In one embodiment, the modulated dendritic cells can be used to assess substances for their potential to provide immuno-therapy and/or anti-tumor effect.

In one embodiment, the present invention provides a method to measure IDO expression and/or enzymatic activity in a dendritic cell to predict the ability of a tumor in a patient to mediate immune evasion. This method can also involve measuring the expression of COX-2 in the cell, whereby overexpression of COX-2 in the cell relative to a control cell, e.g., a cell which is not able to evade the immune system or a normal non-tumoral cell, indicates that the tumor will evade the immune system. This method can be combined with assessing the expression and/or activity of IDO as a predictor of the ability of a tumor to evade the immune system.

In another embodiment, the invention provides a method to treat an overexpressed COX-2 tumor in an individual by administering at least one IDO inhibitor

In another embodiment, the invention provides a method to inhibit the expression of COX-2 in tumor cells and/or treat a tumor with overexpressed COX-2 by contacting the tumor cells with a systemic IDO inhibitor, for example, an inhibitor of the tryptophan catabolism pathway. COX-2 overexpressing tumors include, for example, prostatic carcinomas, colorectal carcinomas, pancreatic carcinomas, cervical carcinomas, endometrial carcinomas, gastric carcinomas, glioblastomas, non-small-cell lung carcinomas, bladder carcinomas, ovarian carcinomas, head and neck carcinomas, esophageal carcinomas, esophageal carcinomas, mesotheliomas, renal cell carcinomas, melanomas, breast carcinomas, thyroid carcinomas, lymphomas, small-cell lung carcinomas, sarcomas, hepatocarcinomas, adrenal carcinomas, choriocarcinomas, cutaneous basocellular carcinomas, and testicular seminomas (see also, Uyttenhove et al (2003) Nat Med 9(10):1269-1274, which is incorporated herein by reference).

The present invention also provides a method for screening for IDO inhibitors. A screening system to identify substances that inhibit IDO and/or modulate the differentiation, proliferation and/or maintenance of dendritic cells can be setup in the basic way of adding substances (for example from a large small molecule library) to cells, and measuring the expression levels and/or the activity level of IDO in the cells before and after the substance is added to the cell. Measuring the levels of IDO gene expression and/or enzymatic activity can be performed by the methods described herein. This process can also be automated using various computer-based technologies and/or automation devices.

The substance(s) identified above can be synthesized by any chemical or biological method. The substance(s) identified above can be prepared in a formulation containing one or more known physiologically acceptable diluents and/or carriers.

To monitor IDO and/or COX-2 expression one can use any conventional method of measuring mRNA levels, including, membrane blotting, PCR, primer extension, RNase protection, and others. In one embodiment, a real-time quantitative RT-PCR (qPCR) is used with Western blot assays. Briefly, RNA is extracted from the various cell populations and cDNA is synthesized using reverse transcriptase. IDO specific RNA is quantified with GAPDH expression serving as an internal control. Real-time amplification and dissociation curves of increasing amounts of total PBMC cDNA using, for example, a SYBR green amplification kit and the IDO specific primers can be used to validate the assay. Dissociation curves can be used to display a single peak, ruling out the presence of primer dimers or parasitic products.

IDO activity may be assayed by quantifying tryptophan catabolism as well as the generation of kynurenine (the catabolite in the pathway). Cells of interest are cultured in HBSS containing a known amount of tryptophan for 4 hours. The amino-acid concentration is estimated by high pressure liquid chromatography (HPLC) using a reversed phase C2/C18 column. The two products can be separated and the area under the curve generated from the chromatography analysis correlates with the actual amount of product. L-tryptophan, is detected at both 254 and 280 nm, while kynurenine triggers peak at 254 nm but no 280 nm, due to hydrolysis of the aromatic ring. Based on titration curves with know concentrations of substrate, we find that reporting kynurenine production based on the 254 nm peak provides us the best curve fit.

In another embodiment, the invention provides a method of treating an immunological disease by administering to the patient a systemic IDO inhibitor, for example, an inhibitor of the tryptophan catabolism pathway, in amount sufficient to treat the immunological disease in the patient. Additionally, the invention provides a method for treating an immunological disease by administering the mature dendritic cells described herein to the patient in an amount sufficient to treat said immunological disease.

In another embodiment, the invention provides a method of treating a patient suffering from a tumor by administering to the patient a systemic IDO inhibitor, for example, an inhibitor of the tryptophan catabolism pathway, in amount sufficient to treat the tumor in the patient. Additionally, the invention provides a method for treating the individual suffering from a tumor by administering the mature dendritic cells described herein to the patient in an amount sufficient to treat said patient.

In another embodiment of the present invention, a method of treating a chronic inflammatory skin condition is provided. In this method, a topical preparation of PGE-2, analogs of PGE-2 or agonists of EP2 are applied to the skin in an amount sufficient to treat the inflammatory skin condition. The topical application of such a preparation can facilitate the upregulation of IDO. Many types of inflammatory skin conditions can be treated in this way and include, for example, autoimmune disorders with skin manifestations (e.g., psoriasis, SLE, scleroderma), allergic conditions (e.g., atopic dermatitis eczema) and chronic graft vs. host disease. In a preferred embodiment, the topical preparation contains PGE-2 or a PGE-2 analog that signals via the EP2 receptor. Additionally, the mature dendritic cells described herein to can be administered to the patient in an amount sufficient to treat the inflammatory skin condition.

In another embodiment of the present invention, preparations of PGE-2, analogs of PGE-2 or agonists of EP2 are formulated as an aerosol preparation for the treatment of inflammatory lung conditions. In this method, the preparation of PGE-2, analogs of PGE-2 or agonists of EP2 can be aerosolized into the lungs in an amount sufficient to treat the inflammatory lung condition. The aerosolized application of such a preparation can facilitate the upregulation of IDO. Many types of inflammatory lung conditions can be treated in this way and include, for example, rheumatologic disorders with respiratory tract manifestations (e.g., SLE, sarcoid, MCTD), allergic or drug-induced conditions mediated by the adaptive immune system (e.g., BOOP) and chronic graft vs. host disease. The preferred aerosolized preparation is PGE-2 or a PGE-2 analog that signals via the EP2 receptor. Additionally, the mature dendritic cells described herein to can be administered to the patient in an amount sufficient to treat the inflammatory lung condition

In another embodiment, the inhibitor preparation is acid stable for delivery into the intestinal tract. In a preferred embodiment, the preparation is acid stable with release into the terminal ileum and/or jejunum for the treatment of an autoimmune disorder with gut/intestinal manifestations (e.g., Crohn's disease, IBD), allergic conditions (e.g., celiac disease) and chronic graft vs. host disease. Additionally, the mature dendritic cells described herein to can be administered to the patient in an amount sufficient to treat the autoimmune disorder with gut/intestinal manifestations.

In the above treatment regimens, the adoptive immunity protocols of the IDO inhibitors during DC maturation can block the negative effects of PGE-2 (IDO upregulation), while preserving the beneficial activity of PGE-2 in stimulating dendritic cell migration.

In the method of treatment, the administration of the inhibitors described herein may be for either “prophylactic” or “therapeutic” purpose. When provided prophylactically, the inhibitors are provided in advance of any symptom. The prophylactic administration of the inhibitors serves to prevent, ameliorate, and/or reduce the severity of any subsequent infection or disease. When provided therapeutically, the inhibitors are provided at (or shortly after) the onset of a symptom of infection or disease. Thus the present invention may be provided to one who is anticipated to develop the disease or after the disease symptoms have presented themselves

By “treating” is meant the slowing, interrupting, arresting or stopping of the progression of the disease or condition and does not necessarily require the complete elimination of all disease symptoms and signs. “Preventing” is intended to include the prophylaxis of the disease, wherein “prophylaxis” is understood to be any degree of inhibition of the time of onset or severity of signs or symptoms of the disease or condition, including, but not limited to, the complete prevention of the disease or condition.

As used herein, the subject patient that would benefit from the administration of the formulations described herein includes any animal which can benefit from these methods. In a preferred embodiment, the subject patient is a human patient.

The inhibitors may be administered in a variety of dosage forms which include, but are not limited to, liquid solutions or suspensions, tablets, pills, powders, suppositories, polymeric microcapsules or microvesicles, liposomes, and injectable or infusible solutions. Similarly, the mature dendritic cells of the present invention can be formulated into a variety of dosage forms and would be typically formulated with at least one pharmaceutically acceptable diluents, carriers, or agents. The preferred form depends upon the mode of administration and the therapeutic application.

The inhibitors and/or mature dendritic cells may be in the form of a liquid, slurry, or sterile solid which can be dissolved in a sterile injectable medium before use. The parenteral administration is preferably intravenously. This injection can be via a syringe or comparable means. This may contain a pharmaceutically acceptable carrier. Alternatively, the compositions may be administered via a mucosal route, in a suitable dose, and in a liquid form. For oral administration, the compositions can be administered in liquid, or solid form with a suitable carrier.

A therapeutically effective amount of the inhibitors and/or the mature dendritic cells for use in the treatment methods when the factors are used either singularly or in combination should be used in an amount that results in the desired effect.

The route of administration can include the typical routes including, for example, orally, subcutaneously, transdermally, intradermally, rectally, vaginally, intramuscularly, intravenously, intraarterially, by direct injection to the brain, and parenterally. In addition, in some circumstances, pulmonary administration may be useful, e.g., pulmonary sprays and other respirable forms.

The various mature dendritic cells, inhibitors, agonists, analogs and/or substances can be administered in a variety of concentrations, forms, etc., which can be optimized by a skilled practitioner, based on a variety of factors of the individual to be treated, including age, gender, weight, height, severity of disease condition, etc.

The invention in one embodiment also provides kits or prepackaged products for the detection of a tumors ability to evade an individual's immune system. Such kits can include one or more of kynurenine, trichloroacetic acid, Erlich reagent, a standard concentration of N-formyl kinyrenine, and a supernatant of a normal (or non-tumor cell) as a control.

The invention also provides a kit for the quantification of mRNA levels of IDO. Such kits can include purified oligonucleotides hybridizing, preferably specifically, to a cDNA encoding IDO and at least one reverse transcriptase amplification tool, reagents, enzymes, etc. Examples of purified oligonucleotides include SEQ ID NO:1, 2, 3, 4, 5, and 6.

In another embodiment, a technical platform comprising a sample of a patients fluid or a solid tumor, a device for quantifying IDO activity, and at least one inhibitor of the stabilizing process of mature dendritic cells having a phenotype that drives CD+8 T cell tolerance. The at least one inhibitor can be selected from IDO inhibitors, PGE2 inhibitors, TNFR inhibitors, TLR inhibitors, and combinations of these.

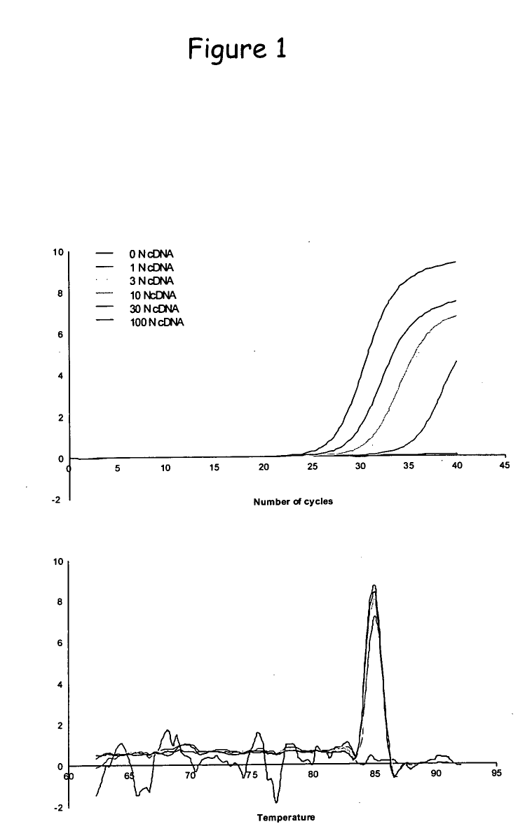

Referring to FIG. 1, amplification plots show an increase in fluorescence from increasing amounts of starting material, total PBMC cDNA. Fewer copies result in more cycles being required for the generation of a fluorescent signal. Following real-time PCR, amplification products were subjected to melting curve analysis, which indicate only one product is present and that primer-dimers did not contribute to the fluorescent signal.

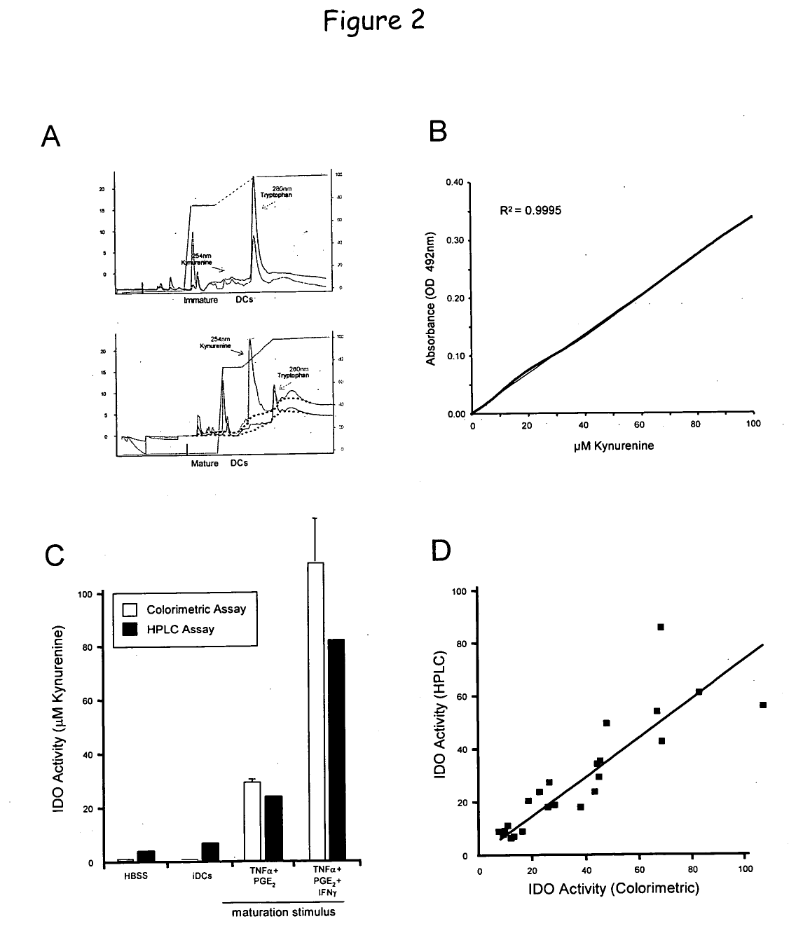

Referring to FIG. 2, HPLC profiles of IDC and mDC (TNFα, PGE2) culture supernatants are shown. Tryptophan, due to its aromatic cycle, is detected at 280 nm while both compounds are at 254 nm. Peaks corresponding to each compound are indicated with arrows. The blue line represents the percentage of elution buffer along time. The dotted lines on the mature DC profile represent baselines used to calculate the area under the peaks. In iDCs, no IDO activity can be detected and the peaks for tryptophan are maximal. mDCs catabolize tryptophan into kynurinine which is eluted at a lower percentage of elution buffer than tryptophan.

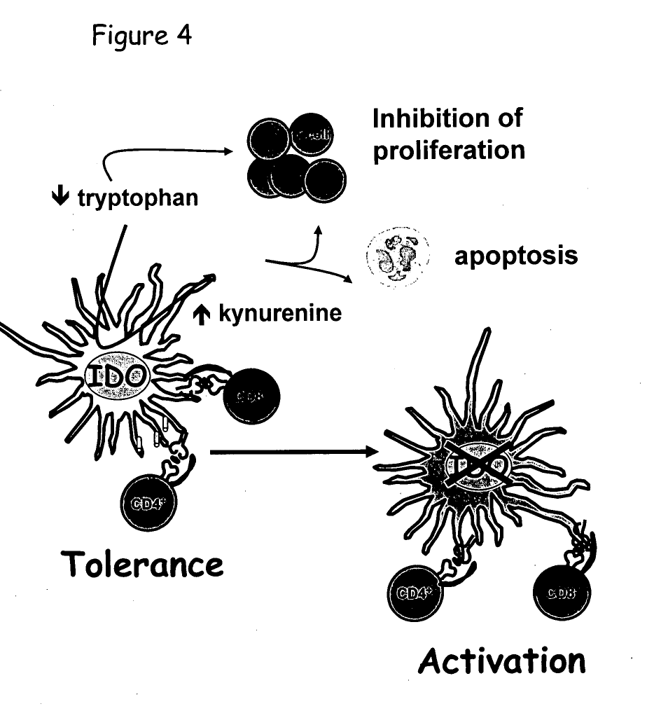

Referring to FIG. 4, IDO, an enzyme involved in tryptophan catabolism has been implicated in T cell tolerance. The scheme displays the currently proposed model in which stimulation of T cells by DC expressing IDO leads to their death by apoptosis (and/or senescence). The presence of cognate help from CD4 T cells, IDO activity is shut down in DC, which leads to CD8 T cell activation. IDO is involved in tryptophan catabolism, plays a role in foeto-maternal tolerance, and may play a role in T cell tolerance. IDO is expressed by mature DC and is induced by g-IFN. IDO activation may occur via B7 reverse signaling.

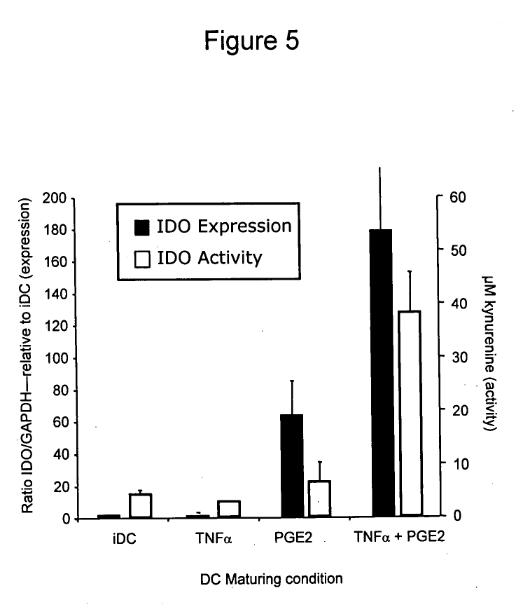

Referring to FIG. 5, PGE2 and TNF-α act synergistically to upregulate indoleamine 2,3-dioxygenase expression and induce its enzyme activity. Human monocyte-derived DC were cultured for 48 hrs with the indicated compounds. At the end of the culture, cells were washed and IDO activity and RNA levels (relative to GAPDH) were assessed as described in material and methods. The graph represents the mean+/−SEM of 2-6 experiments for activity and 3-6 for expression.

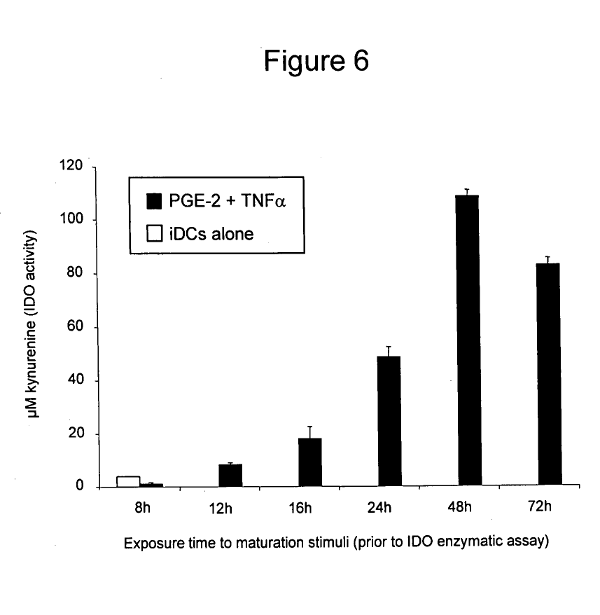

Referring to FIG. 6, IDO activity peaks after 48 hrs of DC maturation. IDO activity was assessed at different time points following the addition of PGE2 and TNF-a. The graph represents mean+/−SD.

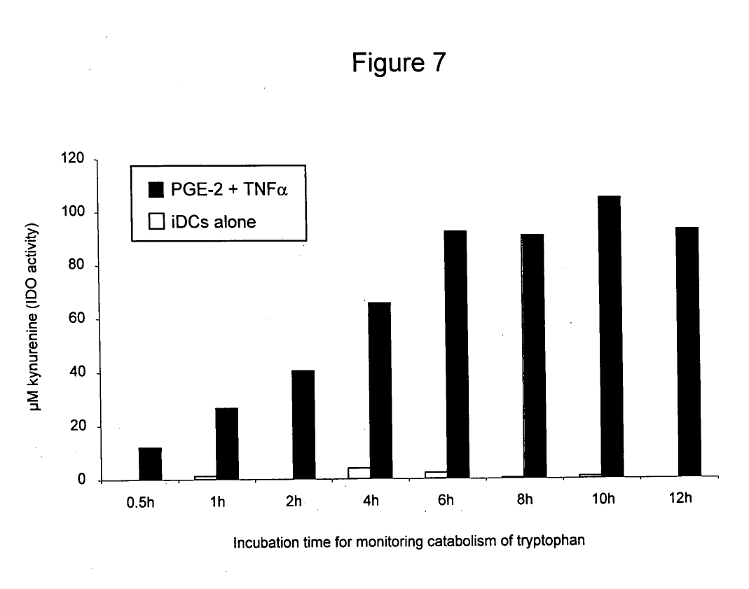

Referring to FIG. 7, PGE2 and TNF-a treated DC catabolize the full transformation of L-tryptophan into kynurenine in 6-10 h. Monocyte-derived DC were cultures for 48 hrs in the presence or absence of PGE2 and TNF-a. At the end of the culture, cells were washed with HBSS and resuspended in a 100 μM L-tryptophan solution. At the end of the indicated periods, supernatants were collected and the concentration of kynurenine assessed using Ehrlich's reagent.

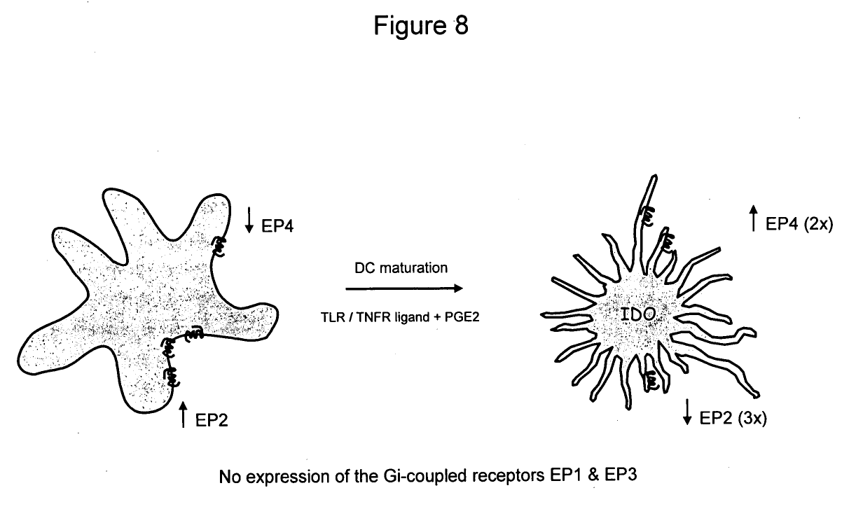

Referring to FIG. 8, EP2 and EP4, the two Ga coupled PGE2 receptors, are expressed on monocyte-derived DC. Amongst the four PGE2 receptors, two (EP1 and EP3) are coupled to Gi proteins and two (EP2 and EP4) are coupled to Ga proteins. Only the latter are expressed on myeloid DC as indicated by our Affymatrix micro-array analysis.

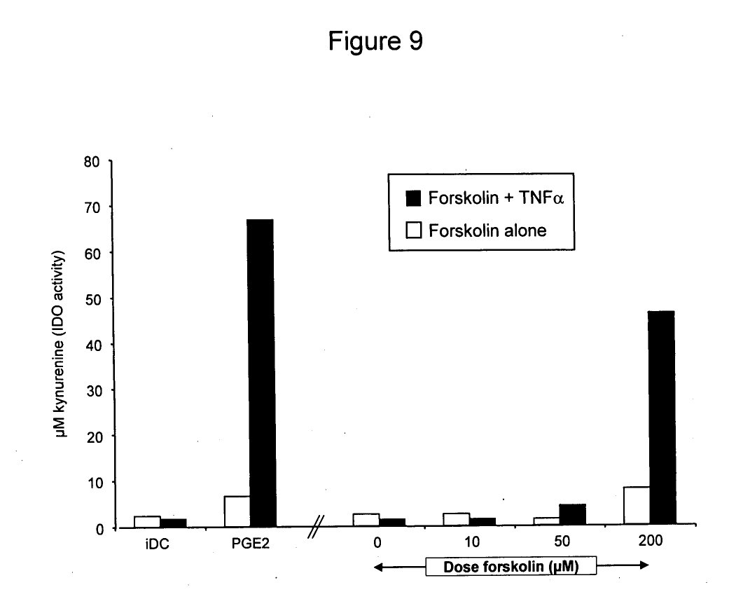

Referring to FIG. 9, the adenylate cyclase activator forskolin mimics PGE2 and induces IDO activity in the presence of TNFa. Monocyte-derived DC were matured during 48 hrs in the presence of increasing doses of forskolin +/− TNFa.

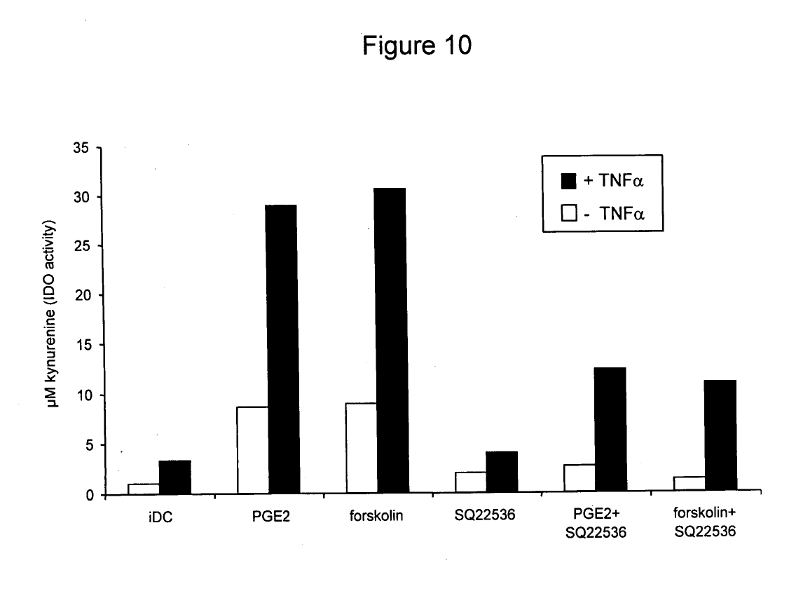

Referring to FIG. 10, The adenylate cyclase inhibitor SQ22536 blocks IDO expression/activity. Monocyte-derived DC were cultured in the presence of PGE₂ or forskolin +/− TNFα for 48 hrs. The effect of inhibiting adenylate cyclase during this culture by SQ22536 was assessed using IDO activity as a bioassay.

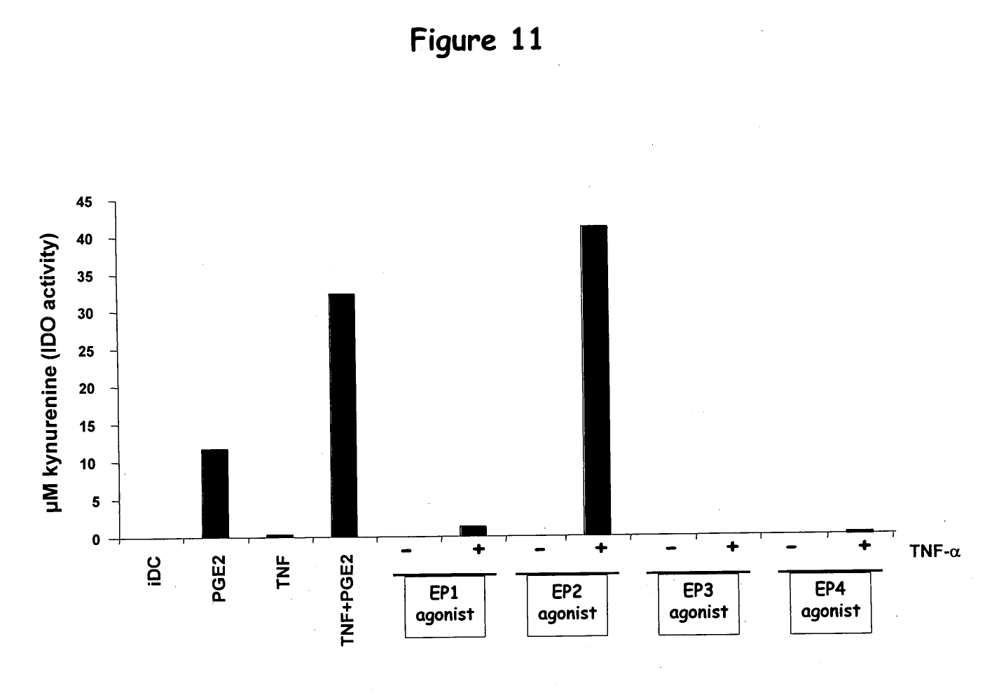

Referring to FIG. 11, the PGE2 receptor EP2 is responsible for stimulating IDO expression in DC. PGE2 was replaced in the DC maturation stimulus by agonists of EP1-EP4. IDO activity was assessed after 48 hrs of maturation.

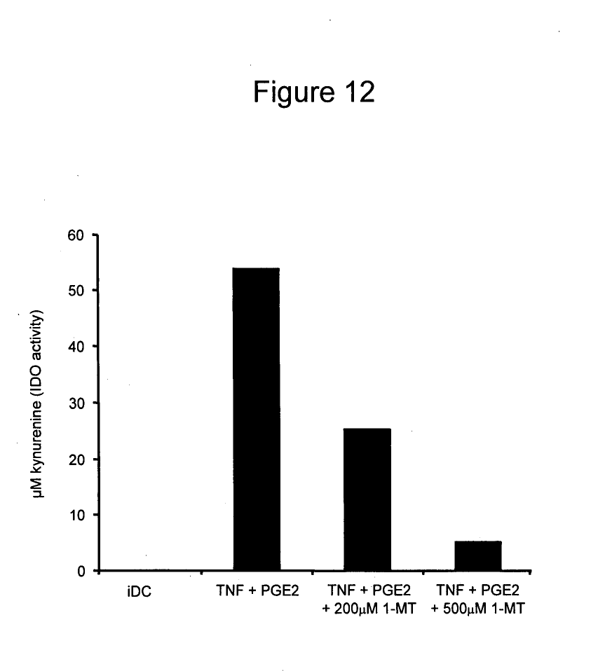

Referring to FIG. 12, IDO activity in mature DC is inhibited by 1-Methyl-D-tryptophan (1-MT). Increasing doses of 1-MT were added to PGE2 and TNF-a during DC maturation and its effect on IDO activity was assessed after 48 hrs.

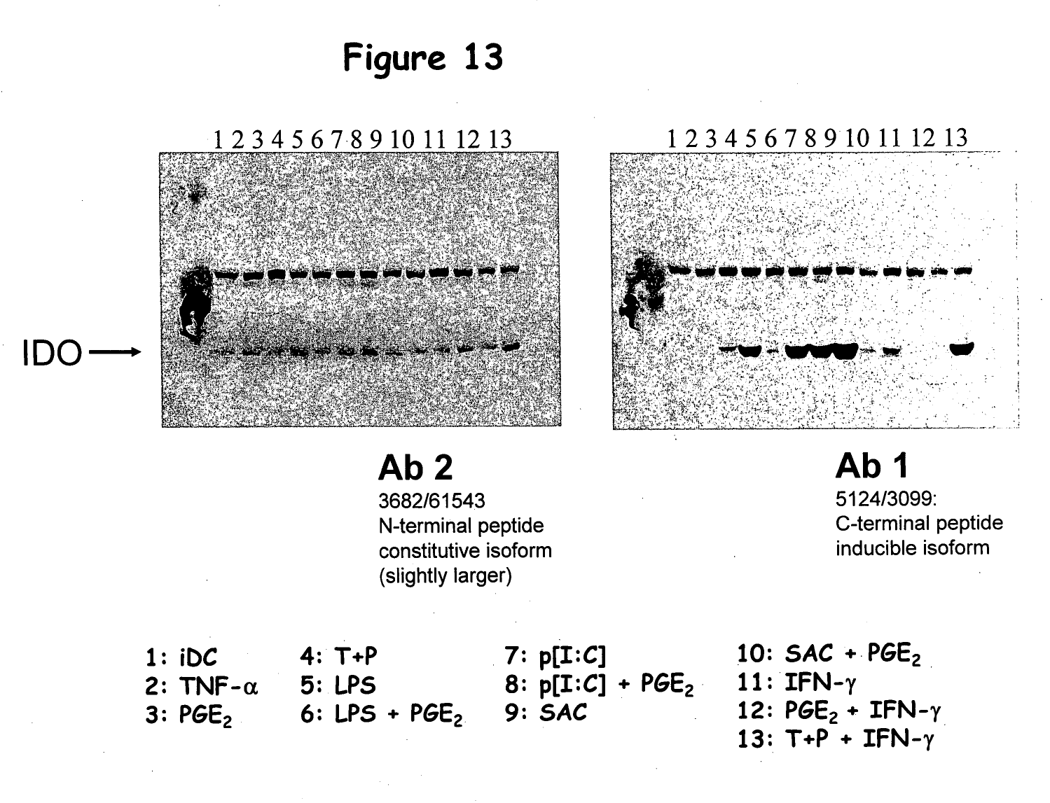

Referring to FIG. 13, PGE2 and TNF-alpha act synergistically to upregulate indoleamine 2,3-dioxygenase active protein levels. Human monocyte-derived DCs were cultured for 48 hrs with the indicated compounds. At the end of the culture, cells were washed and IDO protein expression was assessed by western blot. The left blot is revealed using an antibody raised against the N-terminal peptide of IDO and shows the expression of the constitutive form of the enzyme. The right blot is revealed using an antibody raised against the C-terminal peptide of IDO and shows the expression of the inducible form of the enzyme.

The above written description of the invention provides a manner and process of making and using it such that any person skilled in this art is enabled to make and use the same, this enablement being provided in particular for the subject matter of the appended claims, which make up a part of the original description.

As used herein, the phrases “selected from the group consisting of;” “chosen from,” and the like include mixtures of the specified materials.

Where a numerical limit or range is stated herein, the endpoints are included. Also, all values and subranges within a numerical limit or range are specifically included as if explicitly written out.

As stated above, the above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

Materials and Methods

Human subject materials. Human blood components were obtained from normal donors (EFS, Rungis). Materials were stripped of patient identifiers and shipped to Institut Pasteur in accordance with institutional policy (#HS2003-5720) and the tenets of the Helsinki protocol.

Reagents. TNF α (Endogen, Boston, Mass.) was used at a concentration of 100 ng/ml and PGE2 (Sigma, St. Louis, Mo.) at 5.0 μM, unless otherwise indicated. LPS, serotype 055;B5 (Sigma) was sonicated and used at 50 ng/ml, Staphylococcus aureus Cowan I strain (SAC) was used at 0.0025% wt/vol (Pansorbin, Calbiochem-Behring, LaJolla, Calif.). L-Tryptophan, L-Kynurenine and Forskolin (Sigma) were used as described below; EP 1, EP3 and EP4 agonists (L-335677, L-826266 and L-161982 respectively) were provided by Merck Frost & Co (Canada) and used at a concentration of 50 μM; Butaprost and 19(R)-OH PGE2 (EP2 agonists), sulprostone (EP1>>3 agonist) were obtained from Cayman Chemicals, Ann Arbor, Mich., USA and used at 0.5-250 μM. The adenylate cyclase inhibitor SQ22536 (Biomol international, LP) was titrated and the optimal concentration to inhibit forskolin was found to be 1 mM. H-89 (Sigma) was used at 10-50 μM for the inhibition of protein kinase A (PKA).

Isolation and preparation of cells. Peripheral blood mononuclear cells (PBMCs), DCs and T cells were prepared as described. Briefly, buffy coats were obtained from normal donors (EFS, Rungis) and PBMCs were isolated by sedimentation over Ficoll-Hypaque (Pharmacia Biotech, Piscataway, N.J.). CD 14-enriched and CD-14-depleted fractions were prepared by the use of CD14 Miltenyi microbeads followed by magnetic cell sorting according to the manufacturers' instruction (Miltenyi Biotech, Auburn, Calif.). Immature DCs were prepared by the use of CD14+ fraction by culturing cells in the presence of GM-CSF (Berlix, Seattle, Wash.) and IL-4 (R&D Systems, Minneapolis, Minn.) for 7 days. GM-CSF (1000 U/ml) and IL-4 (500 U/ml) were added to the cultures on days 0, 2, and 4. To generate mature DCs, the cultures were transferred to fresh wells on days 6-7 and the indicated maturation stimulus was added for an additional 1-2 days. At days 6-7, >95% of the cells were CD13⁻CD83⁻HLA-r^{lo} DCs. After maturation, on days 8-9, 70-95% of the cells were of the mature CD13⁻CD83⁻HLA-r^{hi} phenotype. CD4+ and CD8+ T cells were purified from the CD14− fraction to >99% purity by positive selection with the MACS column purification system.

Determination of IDO enzymatic activity and Kynurenine assays. Tryptophan is catabolized by IDO to N-formylkynrenine, which is rapidly converted to kynurenine. Measurement of kynurenine levels is a surrogate marker for IDO activity.

24 h after activation, DCs were washed and resuspended in HBSS containing 100 mM tryptophan (Life Technologies). Except where stated, cells were incubated for an additional 4 hours, followed by harvest of supernatant and quantitation of kynurenine by HPLC or using a colormetric assay.

HPLC was performed according to Young and Lau with minor modifications. Briefly, 40 μl of the clarified sample was injected into a Amersham reverse phase C2/C18 column and eluted with KH₂PO₄ buffer (0.01 M KH2PO4 and 0.15 mM EDA, pH 5.0) containing 10% methanol at a flow rate of 1.0 ml/min. The spectrophotometer was set at 254 nm to detect both kynurenine and tryptophan. Retention time was determined empirically using standard solutions of kynurenine and tryptophan. IDO activity was expressed as the concentration in micromolars of kynurenine in the sample, converted from tryptophan by IDO.

Alternatively, kynurenine concentrations in the culture supernatants were measured using a colormetric assay (i.e., spectrophotometrically). 50 μl of 30% trichloroacetic acid was added to 100 μl of the culture supernatant, vortexed, and then centrifuged at 10,000 rpm for 5 minutes. A 75 μl volume of the supernatant was added to 75 μl of Ehrlich's reagent (100 mg of p-dimethylbenzaldehyde, 5 ml of glacial acetic acid) in a microtiter plate well (96-well format). Optical density was measured at 480 nm filter with a Multiskan MS (Labsystems) microplate reader. The values were referred to a standard curve of defined kynurenine concentrations (0-100 m).

Quantitative analysis of IDO mRNA expression. IDO mRNA detection by real-time PCR. Briefly, RNA was extracted by Tri-reagent (Sigma) and cDNA was synthesized from 1-2 μg RNA using oligo dT (Roche) and Superscript reverse transcriptase (Invitrogen) according to manufacturers' instructions. IDO specific mRNA is quantified relative to GAPDH or TBP (TATA box Binding Protein) using the following primers: IDO forward: 5′AGAGTCAAATCCCTCAGTCC-3′ (SEQ ID NO:1), IDO reverse: 5′-AAATCAGTGCCTCCAGTTCC-3′ (SEQ ID NO:2), GAPDH forward: 5′ACTCCACGACGTACTCAGCG-3′ (SEQ ID NO:3), GAPDH reverse: 5′-GGTCGGAGTCAACGGATTTG-3′ (SEQ ID NO:4) TBP forward: 5′-GCACAGGAGCCAAGAGTGAA-3′ (SEQ ID NO: 5); TBP reverse: 5′-TCACAGCTCCCCACCATATT-3′ (SEQ ID NO: 6),Primers for EP receptors are described in Kamphuis et al. Invest Ophthalmol Vis Sci. 42, 3209-3215 (2001). Quantitative RT-PCR was performed using the SYBR® Green JumpStart™ Taq ReadyMix™ (Sigma) according to manufacturer's instructions. The reactions were run on a PTC200 equipped with a Chromo4™ detector (MJ Research, Mass., USA). The analyses were performed with the Opticon Monitor™ software version 2.03. All the measures were performed in duplicates and validated when the difference in Ct between the 2 measures was less than 0.3. Amplification and dissociation curves of increasing amounts of total PBMC cDNA allowed validation of our assay; and dissociation curves displayed a single peak, ruling out the presence of primer dimers or parasitic products (FIG. 1, top). The ratio gene of interest/housekeeping genes was calculated according to the formula: ratio=2^{-dCt} (dCT=mean Ct gene−mean Ct housekeeping). GAPDH and TBP were used to normalize for IDO and EP-receptor mRNA expression, respectively.

Detection of IDO protein expression. Cell lysates were prepared from 10⁶ DCs using RIPA buffer (20 mM Tris pH7, 5, 150 mM NaCl, 10% Glycerol, 1% Nonidet P-40, Complete® (Roche, protease inhibitor cocktail)). One third of the total protein lysate was separated on 12% or 14% SDS-PAGE. After transfer to PVDF membranes, protein loading was monitored using Ponceau Red staining. IDO protein was detected using a rabbit polyclonal Ab preparation (Munn DH et al. Science 297, 1867-1869 (2002) and Supplemental data of this article) and anti-rabbit IgG-HRP (Santa Cruz Biotechnology, Santa Cruz, Calif.), and visualized by chemiluminescence (ECL, Amersham).

Results

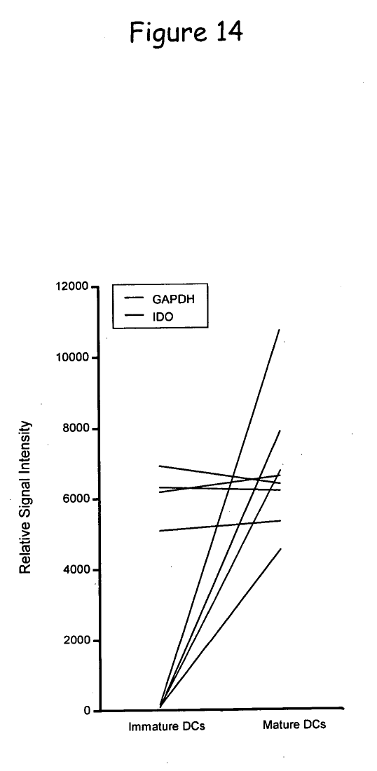

IDO expression and activity are upregulated during DC maturation. The phenotypic and functional changes that occur during DC maturation are critical for generating MHC/peptide complexes and engaging T cells, however, the molecular definition of distinct maturation programs has only recently been considered. As previously reported by others (Huang et al. Science. 294, 870-875 (2001)), the present inventors analyzed the transcriptional profile of monocyte-derived DC at the various stages of differentiation using Affymetrix gene arrays (Longman and Albert, unpublished data). Strikingly, a >100 fold increase in expression of indoleamine 2,3-dioxygenase (IDO) was observed as a result of DC maturation. This has been reproduced in 4/4 donors and the relative signal intensity of IDO mRNA expression is shown, using GAPDH expression as an internal reference (FIG. 14).

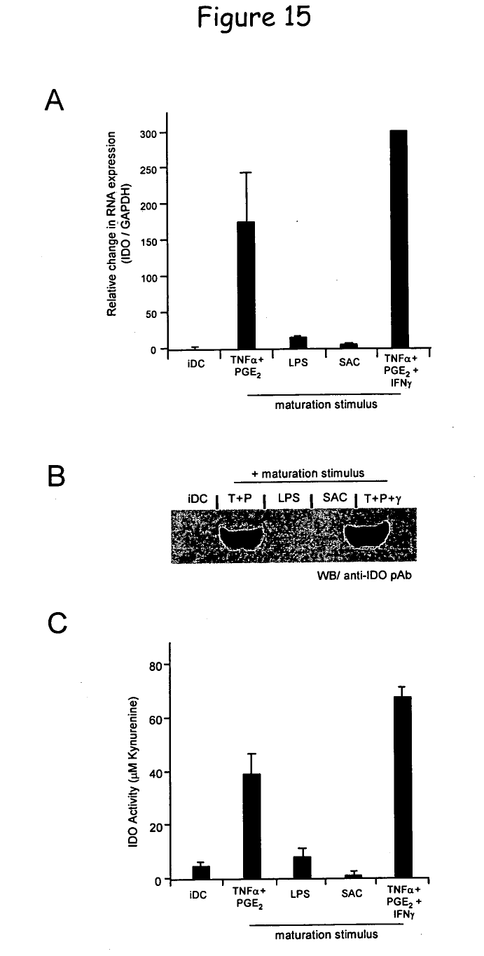

Based on IDO's proposed role in immune tolerance, the present inventors evaluated the effect of different DC maturation stimuli on IDO expression. Despite some published data in this area (Fallarino et al. Int Immunol. 14, 65-68 (2002); Hwu et al. J Immunol. 164, 3596-3599 (2000); Munn et al. Science. 297, 1867-1870 (2002)), a thorough assessment of the expression and activity of IDO as influenced by DC maturation had yet to be performed. The present inventors first established assays to monitor IDO mRNA expression using realtime quantitative RT-PCR (qPCR), and Western blot. RNA was extracted from immature DCs (iDC) exposed to distinct maturation stimuli and IDO expression was quantified as described in the Materials and Methods. Consistent with our chip studies, iDCs matured with TNFα and PGE₂ upregulated IDO mRNA (FIG. 15A). In contrast, iDCs exposed to LPS or SAC did not express measurable levels of IDO mRNA. Evaluation of cell lysates using an IDO rabbit pAb (Munn DH et al. Science 297, 1867-1869 (2002) and Supplemental data of this article) showed good correlation between protein expression and the mRNA levels (FIG. 15B).

It was important to monitor IDO enzymatic activity as there has been a reported difference between IDO expression and activity, suggesting possible post-translational regulation of the enzyme (Fallarino et al. Int Immunol. 14, 65-68 (2002); Grohmann et al. J Immunol. 171, 2581-2587 (2003)). IDO activity may be assayed by quantifying tryptophan catabolism as well as the generation of kynurenine (the first catabolite in the metabolic pathway). After 36-48 hr of exposure to the distinct maturation stimuli, the DCs were cultured in HBSS containing 100 μM tryptophan. After 4 hrs, the concentration of tryptophan and kynurenine was determined by high-pressure liquid chromatography (HPLC). As shown, the two products can be easily separated and the area under the curve correlates with the amount of the respective analyte (FIG. 2A). Notably, the present inventors observed robust enzyme activity in DCs matured with TNFα and PGE₂ but no evidence of tryptophan catabolism was detected in iDCs, LPS- or SACmatured DCs (FIG. 15C). In our initial studies, IFNγ exposed DCs served as a positive control for IDO expression and activity. DCs matured from multiple individuals permitted evaluation of donor variability (Table 1).

TABLE 1
IDO expression and activity

| | IDO mRNA (qPCR)^{†} | | | | IDO activity (μM Kynurenine, HPLC) | | | |
|---|---|---|---|---|---|---|---|---|
| Conditions | n | average | range | SEM | n | average | range | SEM |
| iDC | 7 | 0.017 | 0-0.06 | 0.007 | 6 | 4.3 | 2.8-7.7 | 0.8 |
| TNFα + PGE2 | 7 | 3.3 | 1.01-8.17 | 1.01 | 6 | 38 | 19.3-68.9 | 7.6 |
| LPS | 4 | 0.24 | 0.09-0.39 | 0.06 | 3 | 7.6 | 2.8-14 | 3.3 |
| SAC | 3 | 0.05 | 0-0.13 | 0.04 | 1 | 4.3 | n/a | n/a |
| TNFα + PGE2 + IFN-γ | 1 | 5.1 | n/a^{¥} | n/a | 3 | 66.5 | 60.4-74 | 4.0 |

^{†}Reported as a ratio of relative mRNA expression IDO/GAPDH. Non-normalized data is presented.
^{¥}n/a = Not applicable.

Due to the present inventors interest in monitoring IDO functional activity in several conditions for DC stimulation, they took advantage of a medium-throughput (96-well assay) calorimetric assay for monitoring kynurenine (Grant et al. J Virol. 74, 4110-4115 (2000)). To validate this approach, the present inventors established a standard curve for quantifying kynurenine and compared the experimental values obtained using the calorimetric assay with those from the HPLC analysis. A strong correlation was observed (FIG. 2B-D), thus allowing us to utilize the calorimetric assay for monitoring IDO activity.

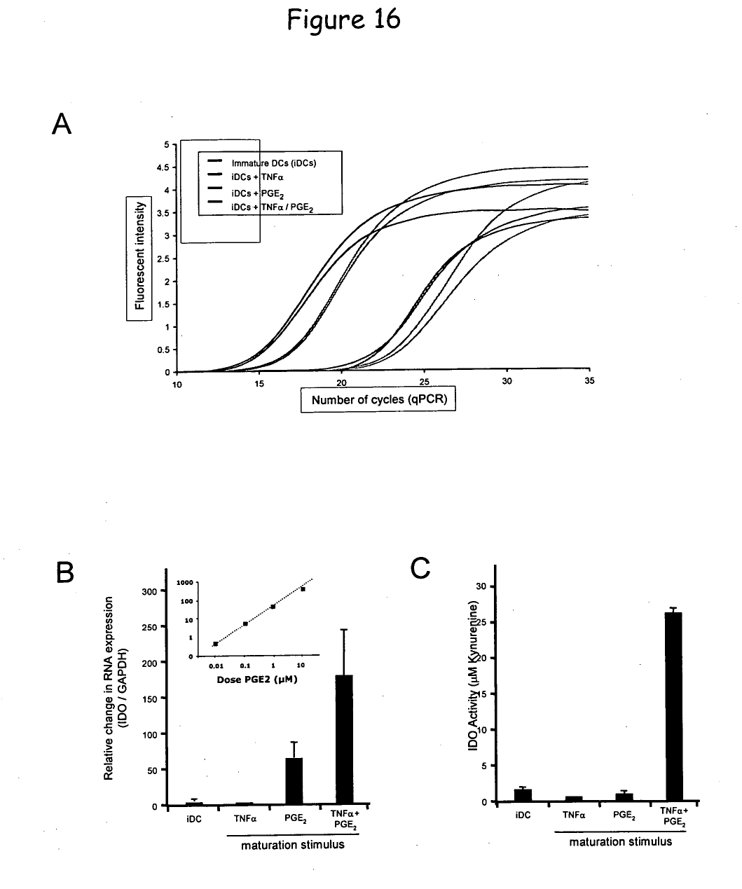

Prostaglandin E₂ is responsible for the expression of IDO mRNA. In an attempt to define the stimuli responsible for IDO expression, the present inventors revealed a surprising and unexpected result. Similar to treatment of iDCs with LPS or SAC, when used alone TNFα did not upregulate IDO expression. Instead, it was exposure of PGE2 that accounted for expression of IDO mRNA (FIG. 16A, B). While the present inventors observed a 50-200 fold increase in transcription of IDO, PGE2 treated DCs lacked measurable IDO activity (FIG. 16C).

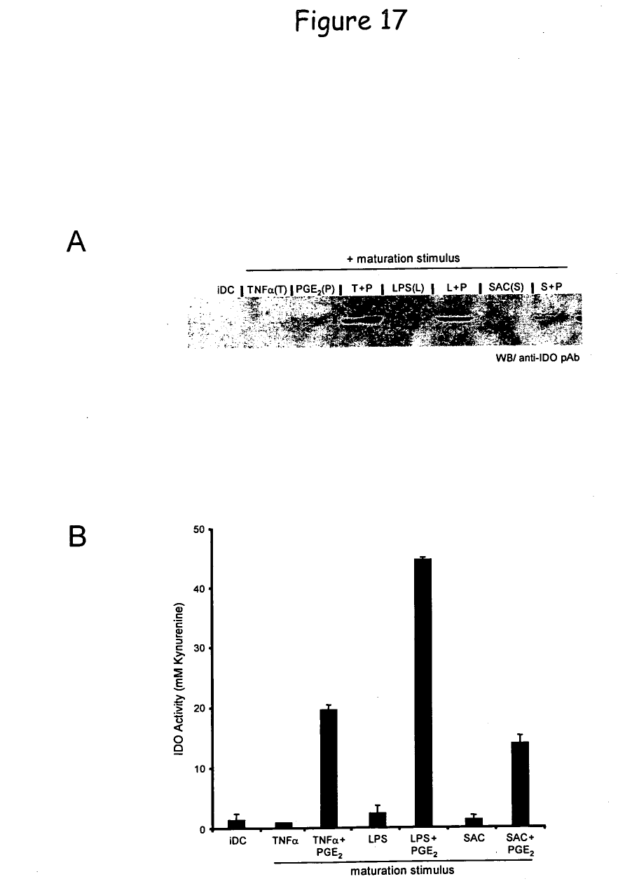

This finding suggests that while PGE2 induces transcription of the IDO gene, a second signal, such as exposure to TNFα, is required to achieve active IDO enzyme. Similar to TNF-R engagement, TLR ligation also induced IDO activity when used in combination with PGE2 (FIG. 17A, B). Characterization of the pAb used suggests that it may be selective for active enzyme (data not shown); consequently, the present inventors are unable to determine if TNF-R/TLR-regulates a transcriptional or post-transcriptional event. What is clear from our data, based on FIGS. 15-17, is that there exists a two-step regulation of IDO activity during DC maturation.

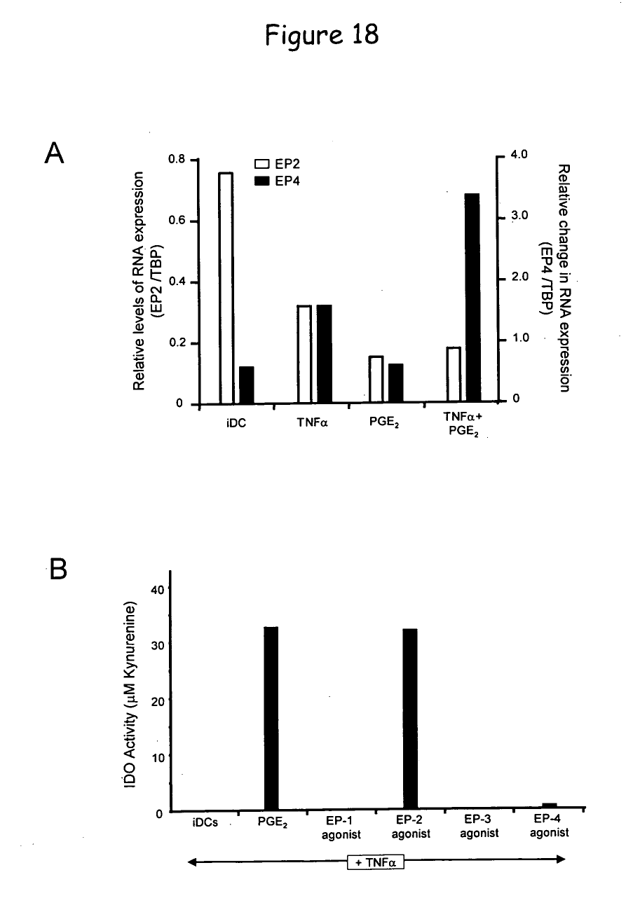

Signaling via EP2 triggers IDO expression. Eight human prostanoid receptors have been described, of which four bind PGE2 with high affinity (Kd in the low nM range). Of these four receptors, EP1 and EP3 are coupled to inhibitory G proteins; whereas EP2 and EP4 signal via stimulatory G proteins. Based on our micro-array data and published studies (Baratelli et al. J Immunol. 173, 5458-5466 (2004)), only EP2 and EP4 are expressed by monocyte-derived DCs (data not shown). The present inventors validated these findings in our culture system using quantitative RT-PCR. As shown, iDCs express higher levels of EP2 and low levels of EP4. After exposure to TNFα and PGE2, the pattern of expression is reversed (FIG. 18A). Of note, PGE2 seems to be responsible for the down-regulation of EP2, whereas both TNFα and PGE2 are required to observe upregulation of EP4.

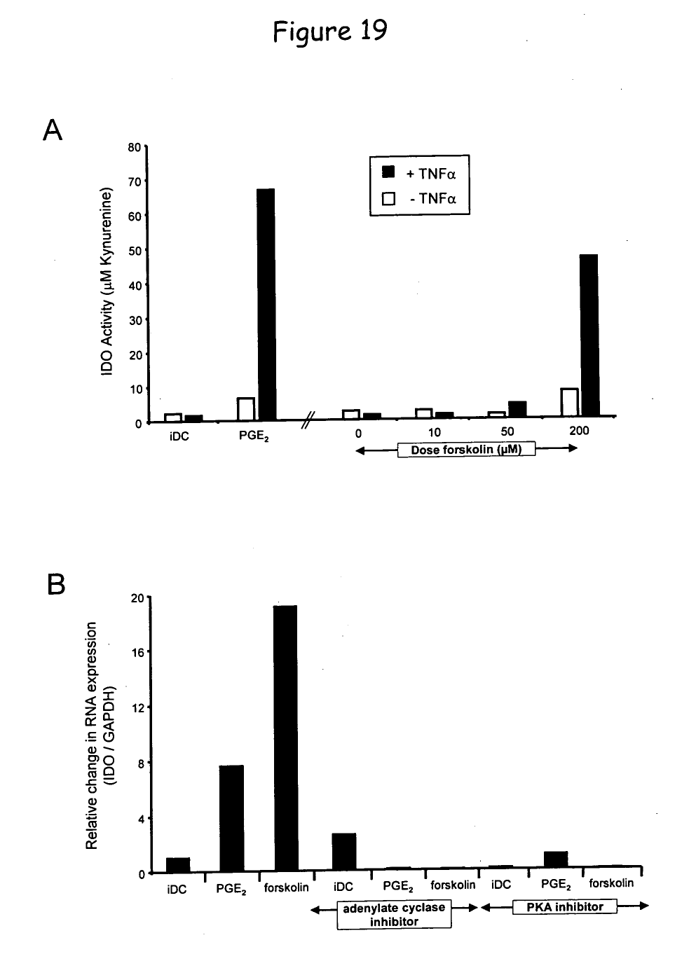

Next, the present inventors evaluated which EP receptor mediates IDO expression. This was done functionally, by exposing iDCs to agonists specific for EP1-4 in the presence of TNFα and IDO activity was measured as described above. In 3/3 individuals, only the EP2 agonist butaprost resulted in the induction of IDO activity (FIG. 18B). As EP2 is a Gαs-coupled receptor, the present inventors tested if ectopic activation of adenylate cyclase would mimic the effects of a receptor agonist. When iDCs were treated with the adenylate cyclase activator forskolin in combination with TNFα, the IDO activity was equivalent to that in TNFα and PGE2 matured DCs (FIG. 19A). The present inventors next directly tested the effect of adding either an inhibitor of adenylate cyclase or the cAMP-triggered kinase PKA on the mRNA expression of IDO. As shown, both inhibitors decreased the PGE2- and forskolin-induced expression of IDO. Together, these results suggest that during DC maturation, PGE2 acts through the EP2 receptor expressed on iDC, which in turn activates adenylate cyclase leading to PKA activation and an increase in the transcription of the gene coding for IDO. The presence of a TNF-R or TLR agonist enhances expression and importantly, this second signal facilitates IDO enzymatic activity.

Obviously, numerous modifications and variations of the present invention are possible in light of the above teachings. It is therefore to be understood that within the scope of the appended claims, the invention may be practiced otherwise than as specifically described herein.

1. A mature dendritic cell comprising a reduced level of IDO activity relative to a normal dendritic cell and which can abolish T cell tolerance in an individual and which drives CD8+ T cell activation.

2. A method of obtaining a mature dendritic cell, comprising contacting the dendritic cell with at least one inhibitor in an amount sufficient to modulate the maturation of the dendritic cell, wherein the at least one inhibitor is selected from the group consisting of an IDO inhibitor, a PEG2 inhibitor, a TNFR inhibitor, a TLR inhibitor and mixtures thereof.

3. The method of claim 2, wherein the mature dendritic cell a reduced level of IDO activity relative to a normal dendritic cell and which can abolish T cell tolerance in an individual and which drives CD8+ T cell activation.

4. The method of claim 1, wherein the at least one inhibitor is an IDO inhibitor.

5. The method of claim 4, wherein the IDO inhibitor is one or more inhibitors selected from the group consisting of (D) isomer analogue of tryptophan and derivatives thereof.

6. The method of claim 5, wherein the IDO inhibitor is one or more inhibitors selected from the group consisting of 1-methyl-(D,I)-tryptophan, β-(3-benzofuranyl)-DL-alanine (1-MT), β-[3-benzo(b)thienyl]-(D,L)-alanine, and 6-nitro-(D,L)-tryptophan.

7. The method of claim 1, wherein the at least one inhibitor is a PEG2 inhibitor.

8. The method of claim 1, wherein the at least one inhibitor is a TNFR inhibitor.

9. The method of claim 1, wherein the at least one inhibitor is a TLR inhibitor.

10. A method of providing a mature dendritic cell composition to an individual, comprising contacting the dendritic cell composition with at least one inhibitor selected from the group consisting of an IDO inhibitor, a PEG2 inhibitor, a TNFR inhibitor, a TLR inhibitor and mixtures thereof; and thereafter providing the dendritic cell composition to the individual.

11. The method of claim 10, wherein the mature dendritic cell comprises a reduced level of IDO activity relative to a normal dendritic cell and which can abolish T cell tolerance in an individual and which drives CD8+ T cell activation.

12. A method for identifying an IDO inhibitor, comprising
contacting a cell with a substance,
measuring the level of at least one of IDO expression and IDO activity; and
comparing the level of at least on IDO expression and IDO activity in the cell contacted with the substance to a cell not contacted with the substance;
wherein a decrease in at least one of IDO expression and IDO activity in the cell contacted with the substance relative to the cell not contacted with the substance indicates that the substance is an IDO inhibitor.

13. The method of claim 12, wherein the level of IDO expression is measured and compared.

14. The method of claim 12, wherein the level of IDO activity is measured and compared.

15. The method of claim 12, wherein both the level of IDO expression and IDO activity is measured and compared.

16. The method of claim 12, which is performed using at least one automated device.

17. A method of predicting a tumors ability to evade an individual's immune system, comprising measuring the level of IDO activity in a cell isolated from the tumor, wherein a decrease in IDO activity relative to a control tumor cell which cannot evade an individuals immune system indicates that the tumor can evade the individual's immune system.

18. The method of claim 17, which further comprises measuring the level of COX-2 expression in the cell isolated from the tumor, wherein an increase in COX-2 expression relative to a control tumor cell which cannot evade an individual's immune system indicates that the tumor can evade the individual's immune system.

19. A method of treating an immunological disorder in an individual, comprising administering at least one IDO inhibitor to an individual in need thereof, in an amount sufficient to block the negative effects of PGE2 stimulation of IDO, stimulate dendritic cell migration, and treat the immunological disorder.

20. A method of treating an immunological disorder in an individual, comprising administering the mature dendritic cell according to claim 1 to an individual in need thereof, in an amount sufficient to block the negative effects of PGE2 stimulation of IDO, stimulate dendritic cell migration, and treat the immunological disorder.

21. A method of treating a tumor in an individual, comprising administering at least one IDO inhibitor to an individual in need thereof, in an amount sufficient to block the negative effects of PGE2 stimulation of IDO, stimulate dendritic cell migration, and treat the tumor in the individual.

22. A method of treating a tumor in an individual, comprising administering the dendritic cell according to claim 1, in an amount sufficient to block the negative effects of PGE2 stimulation of IDO, stimulate dendritic cell migration, and treat the tumor in the individual.

23. A method of treating an inflammatory skin condition in an individual, comprising topically applying a composition on at least one skin area of the individual, wherein the composition comprises at least one of PGE2, PGE-2 agonist, and EP2, in an amount sufficient to upregulate IDO and treat the inflammatory skin condition.

24. A method of treating an inflammatory skin condition in an individual, comprising administering the mature dendritic cell according to claim 1, in an amount sufficient to treat the inflammatory skin condition.

25. A method of treating an inflammatory lung condition in an individual, comprising administering an aerosol composition into a lung area of the individual, wherein the composition comprises at least one of PGE2, PGE-2 agonist, and EP2, in an amount sufficient to upregulate IDO and treat the inflammatory lung condition.

26. A method of treating an inflammatory lung condition in an individual, comprising administering the mature dendritic cell according to claim 1 into a lung area of the individual, in an amount sufficient to treat the inflammatory lung condition.

27. A method of treating a tumor in an individual, comprising administering at least one IDO inhibitor to the individual in an amount sufficient to treat the tumor in said individual, wherein the tumor comprises an overexpressed COX-2 gene.

28. A kit, comprising kynurenine and one or more reagents suitable for detecting a tumor's ability to evade an individual's immune system.

29. The kit of claim 28, which further comprises one or more of trichloroacetic acid, Erlich reagent, a standard concentration of N-formyl kinurenine, and a sample of a supernatant of a non-tumor cell.

30. A kit, comprising at least one purified oligonucleotide which hybridizes with a polynucleotide encoding IDO, and at least one reverse transcriptase reagent suitable for detecting the mRNA level of IDO in a sample.

31. The kit of claim 30, wherein the purified oligonucleotide comprises SEQ ID NO:1, SEQ ID NO:2, SEQ ID NO:3, SEQ ID NO:4, SEQ ID NO: 5, and/or SEQ ID NO: 6.

32. A technical platform, comprising a sample from a patient's fluid or a solid tumor, a device for quantifying IDO activity, and at least one inhibitor for the stabilizing process of mature dendritic cells having a phenotype that drives CD8+ T cell activation, wherein the inhibitor is an IDO inhibitor, a PGE2 inhibitor, a TNFR inhibitor, a TLR inhibitor, or a combination thereof.


SEQUENCE LISTING (6 sequences)

SEQ ID NO: 1 - DNA, 20 bp, Artificial Sequence
  Synthetic DNA
agagtcaaatccctcagtcc

SEQ ID NO: 2 - DNA, 20 bp, Artificial Sequence
  Synthetic DNA
aaatcagtgcctccagttcc

SEQ ID NO: 3 - DNA, 20 bp, Artificial Sequence
  Synthetic DNA
actccacgacgtactcagcg

SEQ ID NO: 4 - DNA, 20 bp, Artificial Sequence
  Synthetic DNA
ggtcggagtcaacggatttg

SEQ ID NO: 5 - DNA, 20 bp, Artificial Sequence
  Synthetic DNA
gcacaggagccaagagtgaa

SEQ ID NO: 6 - DNA, 20 bp, Artificial Sequence
  Synthetic DNA
tcacagctccccaccatatt